(19) United States
(12) Patent Application Publication
(10) Pub. No.: US 2025/0064597 A1
(43) Pub. Date: Feb. 27, 2025

(54) LORDOTIC EXPANDING IMPLANT

(71) Applicant: Bret Michael Berry, Tallahassee, FL (US)

(72) Inventor: Bret Michael Berry, Tallahassee, FL (US)

(21) Appl. No.: 18/943,859

(22) Filed: Nov. 11, 2024

Related U.S. Application Data
(63) Continuation of application No. 18/118,469, filed on Mar. 7, 2023, now Pat. No. 12,138,175.

(51) Int. Cl. A61F 2/44

(52) U.S. Cl. CPC A61F 2/44; A61F 2310/00011; A61F 2310/00179

(57) ABSTRACT

An interbody implant is provided that may expand lordotically and has an additional plate to secure the implant and its expansion.

### TECHNICAL FIELD

This application is a continuation of U.S. application Ser. No. 18/118,469, entitled “Lordotic Expanding Implant”, filed on Mar. 7, 2023, now U.S. Pat. No. 12, 138, 175, issued on Nov. 12, 2024, the entire disclosure of which is incorporated herein by reference.

### TECHNICAL FIELD

The present disclosure generally relates to interbody devices, and more particularly to interbody lordotic expanding implants.

### BACKGROUND

There are many expandable devices for interbody implantation. Expandable implants are desirable because they minimize the access wound needed for surgery, yet while still getting the resulting spinal correction and fusion. Additionally, since patient anatomy can vary greatly from patient to patient, an expandable device can better accommodate and adapt to the anatomy for a more precise fit, which should lead to better stabilization and fusion. Lordosis is defined as an excessive inward curve of the spine. It differs from the spine's normal curves at the cervical, thoracic, and lumbar regions, which are, to a degree, either kyphotic (near the neck) or lordotic (closer to the low back). The spine's natural curves position the head over the pelvis and work as shock absorbers to distribute mechanical stress during movement. One of the biggest drawbacks is their strength. The many small components needed to create the expansion device can greatly reduce strength and lead to failure.

Accordingly, it is an object of the present invention to relatively small lordotic expanding implant with superior strength.

### SUMMARY OF THE INVENTION

The present invention may include a lordotic assembly comprising an expandable assembly made from implantable material. The present invention may further include a U-shaped body with a distal wall and at least one end plate, wherein the body has solid and porous portions. The distal wall has a plurality of pivot apertures located superior and inferior within the distal wall that attaches to the endplates allowing the endplates to pivot. The at least one endplate has an outer surface defined by a set of teeth to provide fixation to a vertebral body endplate. The present invention may also include a drive screw and drive block and at least one arm assembly with retaining means and a retaining plate attached to the expandable assembly with a locking means to hold the expandable assembly in a desired location.

### BRIEF DESCRIPTION OF DRAWINGS

FIG. 1-Perspective view of the final assembled device;

FIG. 2-Perspective view of unexpanded expandable assembly;

FIG. 3-Lateral view of unexpanded expandable assembly;

FIG. 4-Exploded view of expandable assembly;

FIG. 5-Perspective view of expandable assembly;

FIG. 6-Lateral view expanded expandable assembly;

FIG. 7-Distal view of plate;

FIG. 8-Perspective view of plate being placed into expanded expandable assembly;

FIG. 9-Perspective view of expanded expandable assembly with plate;

FIG. 10-Lateral view of expanded expandable assembly with plate;

FIG. 11-Distal view of expanded expandable assembly with plate; and

FIG. 12-Perspective view of expanded expandable assembly with plate and locking screws.

### DETAILED DESCRIPTION

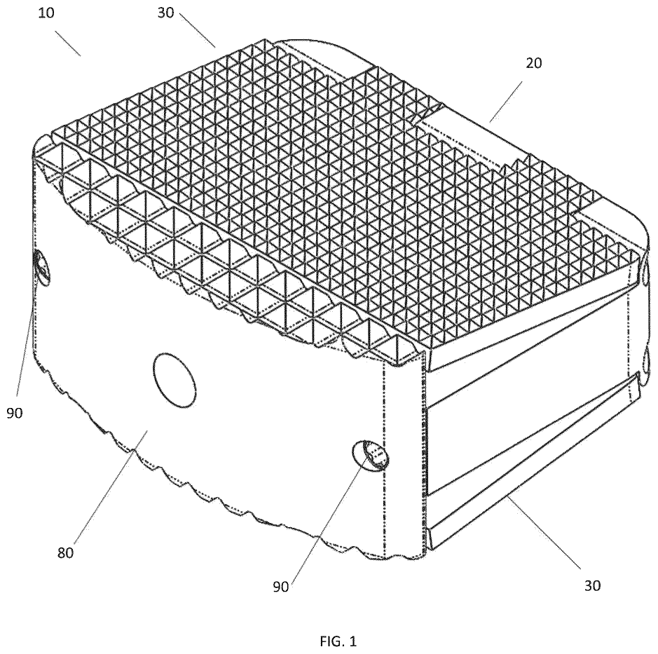

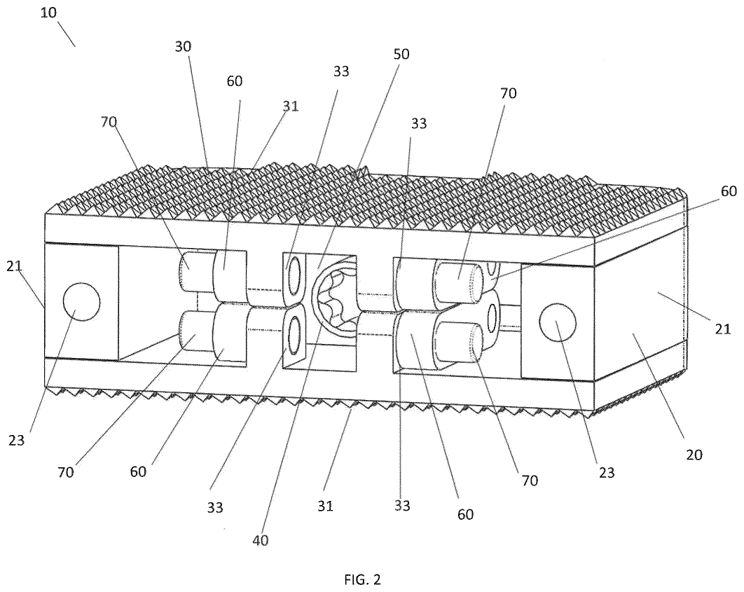

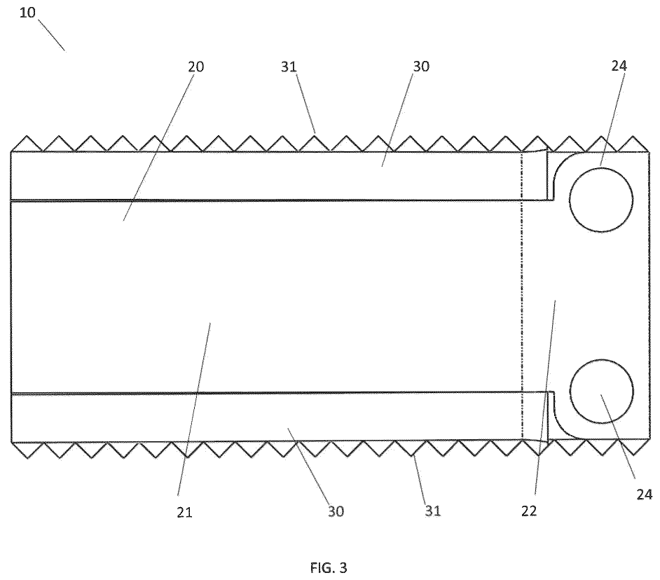

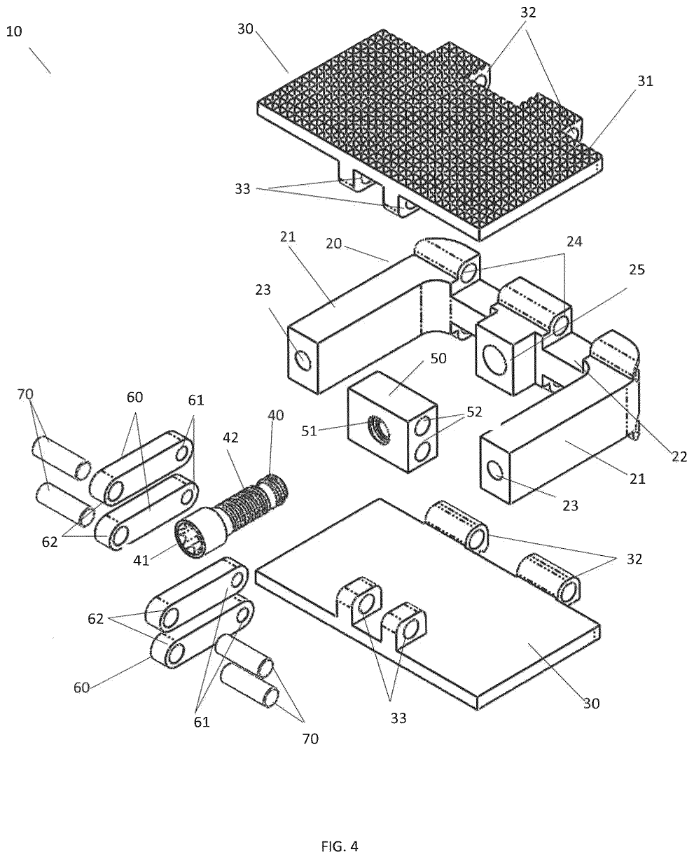

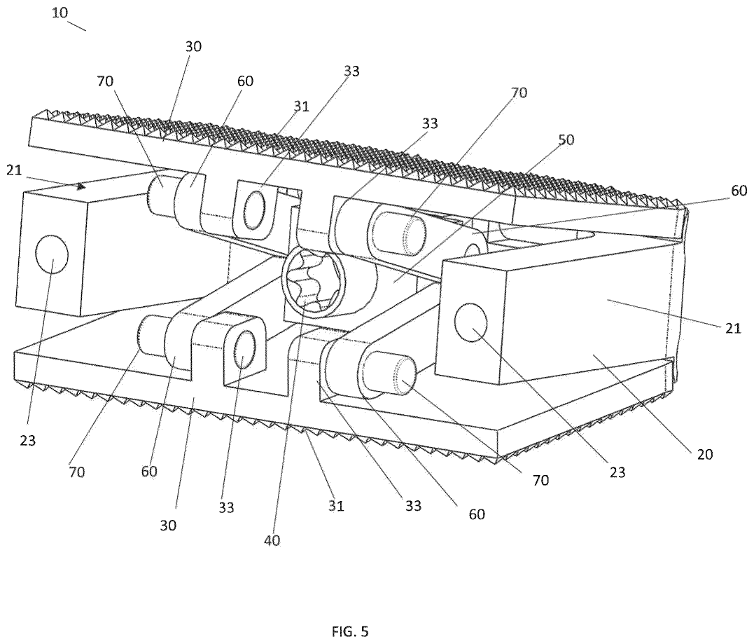

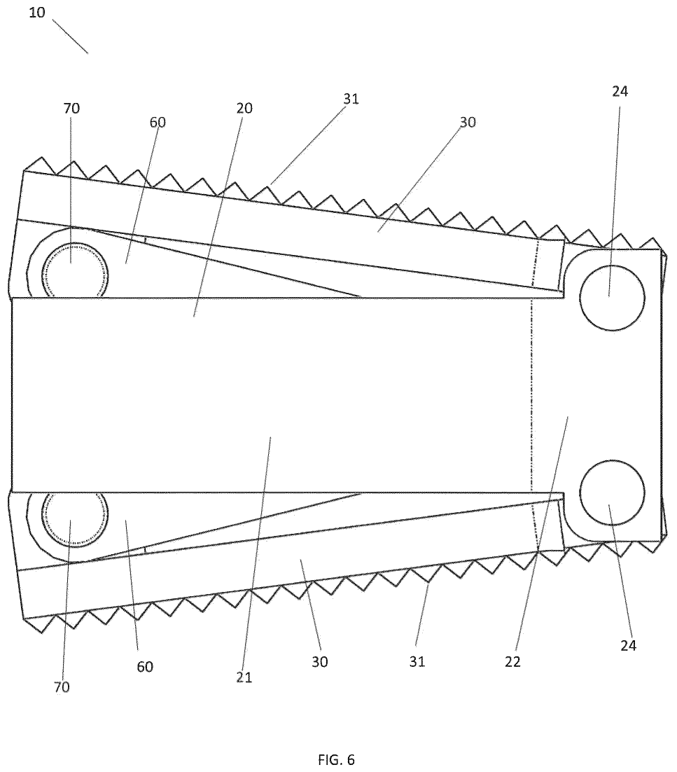

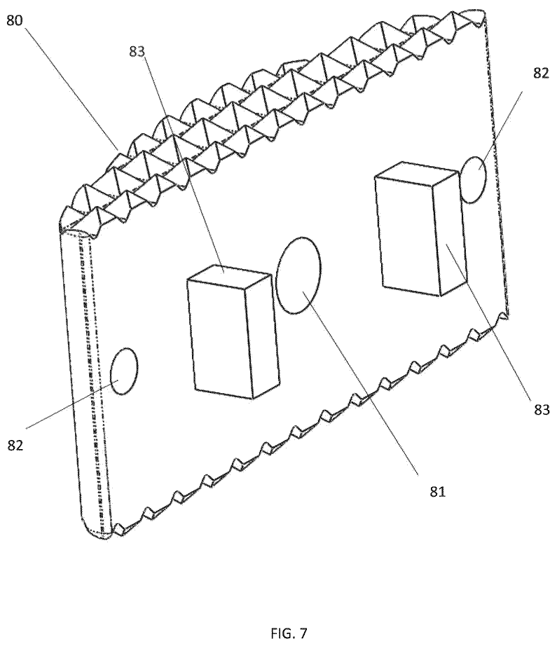

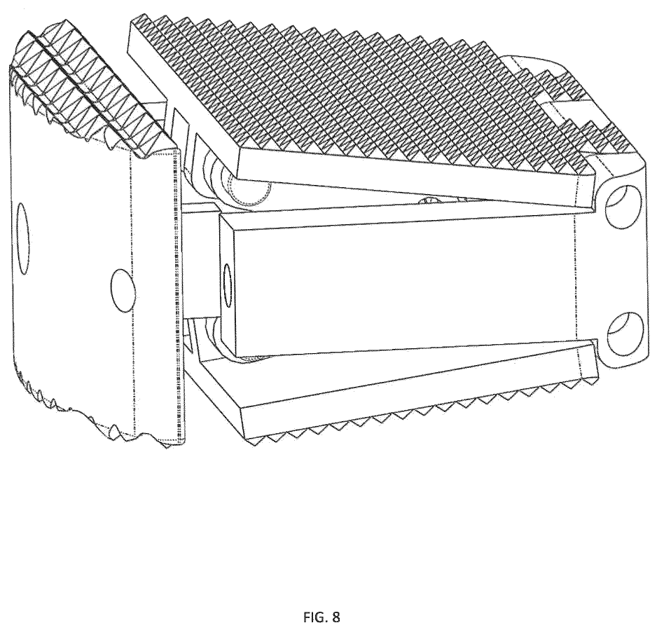

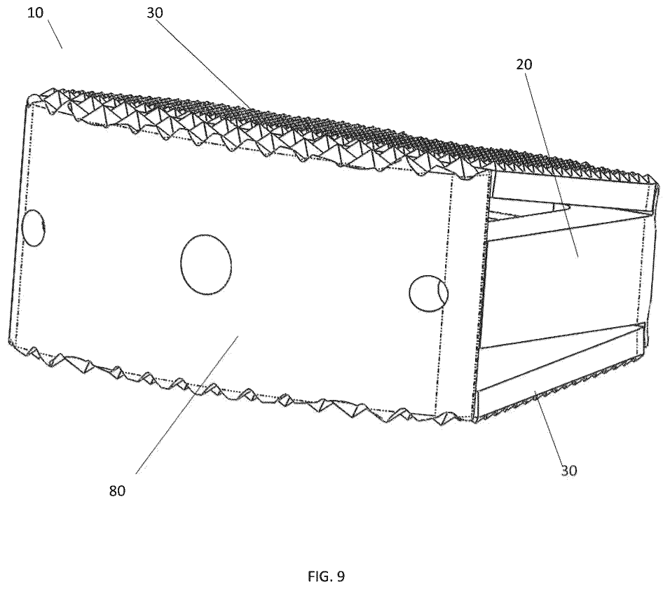

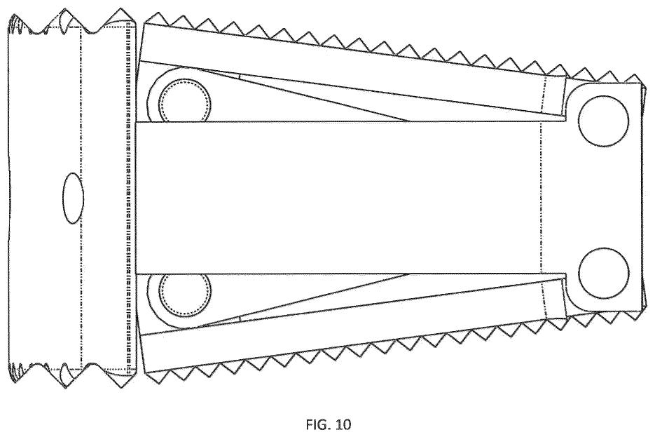

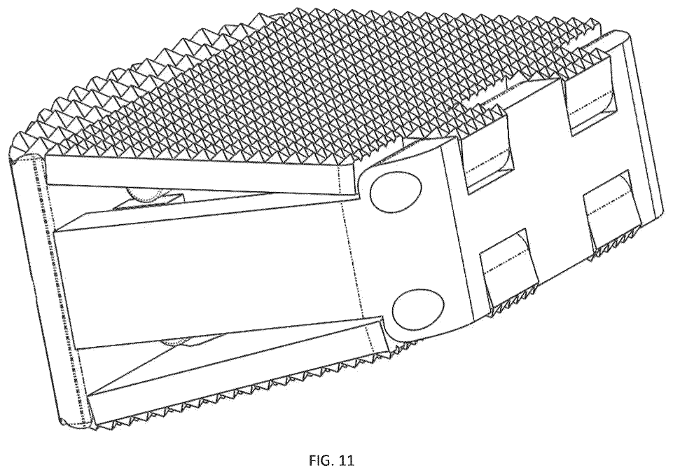

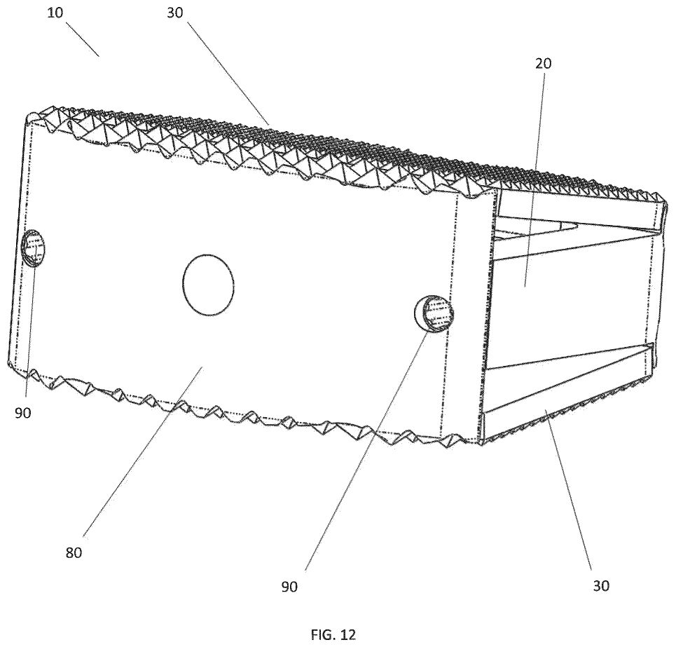

As shown in FIGS. 1-12, the preferred embodiment expandable assembly 10 includes body 20 and at least one endplate 30, drive screw 40, drive block 50, arms 60, and retaining means 70. Retaining plate 80 may be attached to expandable assembly 10 with locking means 90 in order to hold the expansion plate in place.

Body 20 may be a “U” shape in structure, which may be made of an implantable material such as metal, ceramic, or polymer, and may have solid or porous portions. On the distal wall 22 of body 20 are located pivot apertures 24. There are a set of pivot apertures 24 for each endplate 30 to attach to and allows the endplates 30 to pivot about them.

Generally pivot apertures 24 are superior and inferior on distal wall 22. Along the A-P axis of distal wall 22 may be drive aperture 25, which holds drive screw 40, allowing drive screw 40 to spin within it, but retaining drive screw 40 and preventing it from moving forwards or backwards.

On the anterior face of sidewalls 21 of body 20 are attachment apertures 23 for retaining plate 80. Endplates 30 may have an outer surface defined by a set of teeth 31. Teeth 31 are designed to provide fixation to the vertebral endplate. On the distal edge of endplate 30 is distal aperture 32. Distal aperture 32 mates with pivot aperture 24 of body 20 to allow endplate 30 to hinge in reference to body 20. On the proximal edge of endplate 30 is at least one front aperture 33. The front aperture 33 mates with arms 60.

Drive screw 40 mates into drive aperture 25 of body 20. Drive screw 40 consists of threads 42 and drive feature 41. Drive feature 41 faces proximally to allow the surgeon to manipulate drive screw 40. Threads 42 mate with drive block 50.

Drive block 50 may have threaded aperture 51 extending from the proximal to distal faces. Drive screw 40 threads through threaded aperture 51. As drive screw 40 is rotated, drive block 50 moves axially along drive screw 40. In the unexpanded state of expandable assembly 10, drive block 50 is located distally. On the lateral faces of drive block 50 are pivot apertures 52, one pivot aperture 52 per arm 60.

Arms 60 extend from drive block 50 to endplate 30. The block connecting aperture 61 of arm 60 pivots about pivot aperture 52 of drive block 50. Similarly, the endplate connecting aperture 62 of arm 60 pivots about front aperture 33 of endplate 30. As drive block 50 moves forward along drive screw 40, arm 60 also moves forward, thereby forcing the proximal edge of endplate 30 outward. As the distal end of endplate 30 is hinged on the distal wall 22 of body 20, the endplates 30 alter the lordotic angle of the expandable assembly 10. Retaining means 70 hold arm 60 to the front aperture 33 extend out beyond arms 60.

Once endplates 30 of expandable assembly 10 is placed in the desired lordotic angle, retaining plate 80 is put into place. Retaining plate 80 may have an inserter aperture 81 to allow the surgeon to place and manipulate plate 80. Additionally, inserter aperture 81 is large enough to allow drive screw 40 to be accessed through it. Extending proximal to distal on the lateral portions of plate 80 locking apertures 82. Locking apertures 82 align with attachment apertures 23 of body 20. Extending from the distal face of retaining plate 80 are fixation tabs 83. Fixation tabs 83 of plate 80 are aligned with retaining means 70 where they extend past arms 60. Therefore, fixation tabs 83 hold expandable assembly 10 in its expanded position. Lock screws 90 are inserted through retaining plate 80, locking aperture 82 and into attachment aperture 23 of body 20, thereby locking plate 80 to body 20.

It will be apparent to those skilled in the art that various modifications and variations can be made to the disclosed apparatus, system, and method. Other embodiments will be apparent to those skilled in the art from consideration of the specification and practice of the disclosed method and apparatus. It is intended that the specification and examples be considered as exemplary only, with a true scope being indicated by the following claims.

What is claimed is:

1. A lordotic expandable implant comprising:
a body comprising,
a wall having a top face and a bottom face, wherein the top and bottom face are each formed with one or more endplate attachment points,
a first sidewall and second sidewall connected by the wall, wherein the first side wall and the second side wall each extend perpendicularly from the wall such that the body has “U” shaped structure;
a first endplate and second endplate, wherein the first endplate and the second endplate are each configured to attach to and hinge with the one or more endplate attachment points; and
a drive assembly comprising,
a drive block,
a drive screw, and
a plurality of arms, wherein each of the arms connects between the drive block and one of the endplates.

2. The lordotic expandable implant of claim 1, wherein the one or more endplate attachment points are set in from the first sidewall and the second sidewall.

3. The lordotic expandable implant of claim 1, wherein each of the endplates include one or more body attachment points.

4. The lordotic expandable implant of claim 3, wherein each of the endplate attachment points comprises:
a notch formed in the top face or the bottom face of the wall, wherein the notch is configured to receive one of the body attachment points of one of the endplates; and
a pivot aperture on each side of the notch to provide a hinge point for the body attachment point of the endplate, wherein the body attachment point of the endplate is received between the pivot apertures.

5. The lordotic expandable implant of claim 3, wherein each of the body attachment points extends from a rear edge of one of the endplates and each body attachment point of the body attachment points having a top surface that is continuous with a top surface of the endplate from which the body attach point extends.

6. The lordotic expandable implant of claim 1, wherein each of the endplates include one or more drive assembly attachment points.

7. The lordotic expandable implant of claim 1, wherein the drive screw is retained between the first and second sidewall and the drive block moves along the drive screw.

8. The lordotic expandable implant of claim 1, further comprising a retaining plate configured to attach to the body, wherein the plurality of arms are positioned between the retaining plate and the wall when the retaining plate is attached to the body.

9. The lordotic expandable implant of claim 8, wherein the retaining plate is formed with a one or more fixation tabs extending from a rear face of the retaining plate, wherein the rear face of the retaining plate abuts with a front face of the first side wall and a front face of the second side wall when the retaining plate is attached to the body.

10. The lordotic expandable implant of claim 9, wherein the fixation tabs are configured to hold the endplates in an expanded position.

11. A lordotic expandable implant comprising:
a body comprising,
a wall connecting between a first sidewall and a second sidewall, wherein the wall has a top face and a bottom face and each of the sidewalls have a front face that is at a distal end of each of the sidewalls relative to the wall,
one or more endplate attachment points, wherein each of the end plate attachment points is a notch formed in the top face or the bottom face of the wall, and
an attachment aperture formed on the front face of each of the sidewalls;
a first endplate and second endplate, wherein the first endplate and the second endplate are configured to attach to the wall of the body at one of the one or more endplate attachment points;
a drive assembly for expanding the first plate and the second endplate; and
a retaining plate configured to attach to the body via the attachment aperture on each of the sidewalls.

12. The lordotic expandable implant of claim 11, wherein the retaining plate is formed with a one or more fixation tabs extending from a rear face of the retaining plate and one or more locking apertures extending through the retaining plate.

13. The lordotic expandable implant of claim 12, wherein the fixation tabs are configured to hold the endplates in an expanded position.

14. The lordotic expandable implant of claim 11, wherein the retaining plate covers all of a front face of the lordotic expandable implant when the endplates are in an expanded position.

15. The lordotic expandable implant, of claim 11, further comprising one or more body attachment points extending from a rear edge of each of the endplates.

16. The lordotic expandable implant, of claim 11, further comprising one or more drive assembly attachment points extend from an inner surface of each of the endplates.

17. The lordotic expandable implant of claim 11, wherein the drive assembly comprises a drive screw, a drive, block, and a plurality of arms.

18. The lordotic expandable implant of claim 17, wherein each of the arms connects between the drive block and one of the endplates.

19. The lordotic expandable implant of claim 17, wherein the drive block is formed with a threaded aperture through its center that is configured to receive the drive screw.

20. A lordotic expandable implant comprising:
a body comprising,
a first sidewall and a second sidewall, wherein each of the sidewalls has a front face configured with a drive aperture,
a wall connecting between the first sidewall and the second sidewall, wherein the wall has a top face and a bottom face and is formed with a drive aperture, and
one or more pivot aperture pairs formed in the top face and the bottom face of the wall, wherein each pivot aperture of the pivot aperture pairs are separated by a notch form in the wall;
a first endplate and a second endplate, each of the endplates being configured with one or more body attachment points extending from a rear edge of each of the endplates, wherein each of the body attachment points are configured to pivot between one of the pairs of pivot apertures; and
a drive assembly comprising,
a drive screw, wherein the drive aperture is configured to receive a first end of the drive screw to enable the drive screw to rotate within the drive aperture but prevent the drive screw from moving forwards or backwards
a drive block, and
a plurality of arms, wherein each of the arms connects between the drive block and one of the endplates.
